(19) United States
(12) Patent Application Publication
(10) Pub. No.: US 2016/0066891 A1
(43) Pub. Date: Mar. 10, 2016

(54) IMAGE REPRESENTATION SET

(71) Applicant: International Business Machines Corporation, Armonk, NY (US)

(72) Inventors: Ella Barkan, Haifa (IL); Ami Ben-Horesh, Holon (IL); Sharbell Hashoul, Haifa (IL); Andre Heilper, Haifa (IL); Pavel Kisilev, Maalot (IL); Eugene Walach, Haifa (IL)

(21) Appl. No.: 14/481,916

(22) Filed: Sep. 10, 2014

(51) Int. Cl. A61B 8/08

(52) U.S. Cl. CPC A61B 8/5223; A61B 8/0825; A61B 8/085

(57) ABSTRACT

A computer implemented method, a computerized system and a computer program product for image representation set creation. The computer implemented method comprises obtaining an image of a subject, wherein the image is produced using an imaging modality. The method further comprises automatically determining, by a processor, values to an image representation set with respect to the image, wherein the image representation set consists of semantic representation parameters of the image according to the imaging modality and according to a clinical diagnosis problem that is ascertainable from the image, wherein a total number of combinations of values of the semantic representation parameters is below a human comprehension threshold; and determining a decision regarding the clinical diagnosis problem based on the values of the image representation set of the image.

### TECHNICAL FIELD

The present disclosure relates to creation of image representation set in general, and to creation of image representation set for clinical diagnosis, in particular.

### BACKGROUND

Medical imaging is the technique, process and art of creating visual representations of the interior of a body for clinical analysis and medical intervention. Medical imaging seeks to reveal internal structures hidden by the skin and bones, as well as to diagnose and treat disease. Medical imaging may also be used to establish a database of normal anatomy and physiology to make it possible to identify abnormalities.

One example of medical imaging may be ultrasonography which is a technique that is based on ultrasound waves and which helps physicians to visualize the structures of internal organs of human body. Another example of medical imaging may be mammography which is a technique that is based on low-energy X-rays and which may help physicians to early detect breast cancer.

In some cases, computer vision techniques may be utilized to process and potentially extract information from images. Various of features may be extracted from the image, including features which are not coherent to a human observer. The features may be used to classify the images, such as utilizing clustering algorithms or other machine learning techniques.

### BRIEF SUMMARY

One exemplary embodiment of the disclosed subject matter is a computer-implemented method comprising obtaining an image of a subject, wherein the image is produced using an imaging modality. The method further comprising automatically determining, by a processor, values to an image representation set with respect to the image, wherein the image representation set consists of semantic representation parameters of the image according to the imaging modality and according to a clinical diagnosis problem that is ascertainable from the image, wherein a total number of combinations of values of the semantic representation parameters is below a human comprehension threshold; and determining a decision regarding the clinical diagnosis problem based on the values of the image representation set of the image.

Another exemplary embodiment of the disclosed subject matter is computerized apparatus having a processor, the processor being adapted to perform the steps of: obtaining an image of a subject, wherein the image is produced using an imaging modality; determining values to an image representation set with respect to the image, wherein the image representation set consists of semantic representation parameters of the image according to the imaging modality and according to a clinical diagnosis problem that is ascertainable from the image, wherein a total number of combinations of values of the semantic representation parameters is below a human comprehension threshold; and determining a decision regarding the clinical diagnosis problem based on the values of the image representation set of the image.

Yet another exemplary embodiment of the disclosed subject matter is a computer program product comprising a computer readable storage medium retaining program instructions, which program instructions when read by a processor, cause the processor to perform a method comprising: obtaining an image of a subject, wherein the image is produced using an imaging modality; determining values to an image representation set with respect to the image, wherein the image representation set consists of semantic representation parameters of the image according to the imaging modality and according to a clinical diagnosis problem that is ascertainable from the image, wherein a total number of combinations of values of the semantic representation parameters is below a human comprehension threshold; and determining a decision regarding the clinical diagnosis problem based on the values of the image representation set of the image.

### THE BRIEF DESCRIPTION OF THE SEVERAL VIEWS OF THE DRAWINGS

The present disclosed subject matter will be understood and appreciated more fully from the following detailed description taken in conjunction with the drawings in which corresponding or like numerals or characters indicate corresponding or like components. Unless indicated otherwise, the drawings provide exemplary embodiments or aspects of the disclosure and do not limit the scope of the disclosure. In the drawings:

FIG. 1 shows a flowchart diagram of a method, in accordance with some exemplary embodiments of the disclosed subject matter;

FIG. 2 shows an illustration of an image, in accordance with some exemplary embodiments of the subject matter; and

FIG. 3 shows a block diagram of an apparatus, in accordance with some exemplary embodiments of the disclosed subject matter.

### DETAILED DESCRIPTION

One technical problem dealt with by the disclosed subject matter is to provide a mechanism which may be useful to represent an image for both a human practitioner and a computerized device. In some exemplary embodiments, the image may be a medical image of a subject.

In some exemplary embodiments, the human practitioner may be a physician, a doctor or the like. The human practitioner may aim to solve a problem that may be ascertainable from the image. The problem may be a clinical diagnosis problem of the subject. As an example only, the problem may be determining whether a tumor appearing in the image is malignant or benign.

In some exemplary embodiments, the computerized device may be an automatic diagnostic tool, a Computer-Aided Diagnosis (CAD) device, a tool performing computer vision processing of clinical images, or the like.

The image may be produced using a medical imagining tool using any imaging modality. The imagining modality may be, for example, radiography, Magnetic Resonance Imaging (MRI), nuclear imaging, ultrasound, elastography, tactile imaging, photoacoustic imaging, thermography, tomography, echocardiography, functional near-infrared spectroscopy, mammography, or the like.

In some exemplary embodiments, the image may be an ultrasonic image, which may be created by recording the echo of ultrasound waves that is being returned from within a body. The ultrasound waves may reflect and may echo off parts of the tissue being imaged; the echo may be recorded and may be used to generate the ultrasonic image.

The ultrasonic image may be produced using a brightness-mode ultrasound imaging modality, also known as B-mode. Brightness mode may provide structural information utilizing different “brightness” in a two-dimensional image. Such images, also referred to as B-mode images, may display a two-dimensional cross-section of the tissue being imaged. In some exemplary embodiments, other ultrasound imaging modalities may be used, such as for example, A-mode, C-mode, M-mode, Doppler-mode, or the like. In some exemplary embodiments, the ultrasonic image may be a 2D image of a plane of the tissue, a 3D image of the tissue, or the like.

In some exemplary embodiments, the subject may be a human breast, and the clinical diagnosis problem may be detecting breast tumor according to the image of the breast. Additionally or alternatively, the clinical diagnosis problem may comprise determining malignancy of the tumor, i.e. determining whether the tumor appearing in the image (i.e., the subject) is benign or malignant.

In some exemplary embodiments, the image may be a mammographic image, which may be created using low-energy X-rays, to examine the human breast. The mammographic image may be used as a diagnostic and a screening tool, to early detect breast cancer. The detection may be performed through detection of characteristic masses, microcalcifications, or the like.

It will be noted that the disclosed subject matter is not limited to any specific kind of images or any imaging modality. However, for the purpose of clarity and without limiting the disclosed subject matter, the disclosed subject matter is exemplified with respect to ultrasonic and mammographic images.

One technical solution is to automatically determine values to an image representation set with respect to the image. The image representation set may comprise semantic representation parameters of the image. In some exemplary embodiments, the semantic representation parameters may be parameters describing features of the image, and may combine imaging and clinical features. The semantic representation set may differ according to the imaging modality of the image and the clinical diagnosis problem. In some exemplary embodiments, the image representation set may be defined using parameters that are relatively easy for a human practitioner to ascertain from the image. As an example, a median gray level in the image is a feature that may be ascertainable from the image by a computerized device but may be hard, if not impossible, for a human practitioner to determine. In contrast, homogeneity feature of a tumor may be relatively easy for a human practitioner to determine based on an image. By using features that are comprehensible to both human practitioners and computerized devices, the image representation set may define a unified language used by both humans and computers. As an example, the human practitioners may verify that a computerized device has analyzed the image correctly by reviewing the image and the values of the image representation set. As another example, a human practitioner may manually define the repercussions of each potential valuation of the image representation set thereby providing the computerized system with a database for automatic analysis that is based on knowledge of experts and potentially on results from various studies.

In some exemplary embodiments, the semantic representation parameters for the image representation set may be automatically selected, such as using rules, configurations, databases, or the like. The selection may be based on the clinical diagnosis problem. Additionally or alternatively, the automatic selection of the semantic representation parameters may be based on the imaging modality.

In some exemplary embodiments, the number of combination of values of the semantic representation parameters may be below a human comprehension threshold. In the present disclosure, “human comprehension threshold” is a number of combinations that a human practitioner can reasonably distinguish therebetween. The human comprehension threshold may not exceed a number of a thousand combinations as it is unreasonable to expect a human practitioner to be able to categorize one problem into a thousand different combinations (e.g., to know, by heart, what is the impact of each of the thousand combinations). The human comprehension threshold may be dozens of combinations (e.g., 20 combinations, 40 combinations, 60 combinations, or the like), or even a hundred combinations.

For example, given a medical image of a tumor, values to an image representation set consisting ten semantic representation parameters may be determined. The image representation set may consist: a tumor shape parameter, a tumor size parameter, a smoothness parameter, an edge sharpness parameter, a brightness parameter, a homogeneity parameter, an echogenicity parameter, a mobility parameter, an intensity parameter, a stiffness parameter or the like. Each parameter may have four possible values. In this case, the total number of combination of values of the semantic representation parameters may be 4¹⁰=1042576 combinations. For This number of combinations, a human practitioner may not be able to distinguish between the meanings of different image representation sets. Therefore, the number of combinations in this example may be above the human comprehension threshold.

In some exemplary embodiments, the total number of combinations of values of the semantic representation parameters may be greater than ten and below a hundred. For example, given an image of a tumor, the image representation set may consist: a tumor shape parameter, a tumor size parameter, a smoothness parameter, and an edge sharpness parameter. The tumor shape parameter may have three possible values: spherical, oblate or prolate. The tumor size parameter may have four possible values: T₁ (from 0 to 2 centimeters), T₂ (from 2 to 5 centimeters), T₃ (greater than 5 centimeters) or T₄ (a tumor of any size that has broken through the skin). The smoothness parameter may have three possible values: smooth, partially smooth or nonsmooth. The edge sharpness parameter may have two possible values: high acutance or low acutance. The total number of combination of values of the semantic representation parameters may be 3*4*3*2=72 combinations. Therefore, the number of combinations in this example may be below the human comprehension threshold.

In some exemplary embodiments, the total number of combinations of values of the semantic representation parameters may be greater than a dozen and below forty. Referring again to the above mentioned example, in case the image representation set comprises only three semantic representation parameters: a tumor shape parameter, a tumor size parameter and an edge sharpness parameter. In this case, the total number of combination of values of the semantic representation parameters may be 3*4*2=24 combinations, which is below the human comprehension threshold.

In some exemplary embodiments, the image representation set may be defined using parameters that are sufficient to determine a decision regarding the clinical diagnosis problem. The valuation of the image representation set may define a correct answer for the clinical diagnosis problem. Different valuations of the same image representation set may yield different answers or different decisions regarding the clinical diagnosis problem. In some cases, the image representation set may be comprised of a relatively small set of parameters with a relatively small number of total potential combinations, while still providing sufficient information to distinguish between one case and the other for the purpose of providing an answer to the clinical question. In some cases, the image representation set may comprise parameters that are relatively highly relevant to the problem being addressed and whose values have a relatively high likelihood of affecting the answer.

In some exemplary embodiments, a verification that the image representation set is sufficient for diagnostic reasoning may be performed. The verification may include defining different image representation sets for the same kind of images and clinical problems, and authenticating that the image representation sets can be used to yield correct diagnostic answers.

In some exemplary embodiments, when the image is a B-mode ultrasonic image of a tumor within a human breast tissue, the semantic representation parameters may consist of: a Breast Imaging-Reporting and Data System (BI-RADS) parameter, a homogeneity parameter and an echogenicity parameter.

In some exemplary embodiments, BI-RADS parameter may be a quality assurance parameter, which may standardize reporting of medical images, such as ultrasonic images, mammographic images, or the like. BI-RADS parameter may be used to communicate a patient's risk of developing breast cancer. In some exemplary embodiment, BI-RADS parameter may have several assessment categories. The assessment categories may be standardized numerical codes typically assigned by a radiologist after interpreting a mammogram or an ultrasonic image. This may allow for concise and unambiguous understanding of patient records between multiple doctors and medical facilities. In some exemplary embodiments, BI-RADS assessment categories may be chosen from the set of: “0: Incomplete, 1: Negative, 2: Benign findings, 3: Probably benign, 4: Suspicious abnormality, 5: Highly suggestive of malignancy, 6: Known biopsy—proven malignancy”.

In some exemplary embodiment, only four values of BI-RADS parameter may be available for some clinical diagnosis problems. For example, in case the problem is the malignancy of the tumor, the potential values may be: 2, 3, 4 and 5, as BI-RADS of value 0 may stand for low image quality (e.g., image not usable), BI-RADS of value 1 may mean no tumor exist (e.g., there is no tumor to analyze) and BI-RADS of value 6 may indicate diagnosis known a priori (e.g., the tumor is a-priori known to be malignant).

In some exemplary embodiments, the homogeneity parameter may be a parameter describing uniformity of the subject (e.g., tumor). The homogeneity parameter may analyze uniformity of dose distribution in the subject volume within the image. In some exemplary embodiments, the homogeneity parameter may have two values: “Homogenous” or “Heterogeneous”, describing the tumor within the image.

In some exemplary embodiments, the echogenicity parameter may be a parameter describing the ability to bounce an echo, e.g. return a signal in ultrasonic images. In some exemplary embodiment, echogenicity may be higher when the surface bouncing the sound echo reflects increased sound waves. Tissues that have higher echogenicity may be called “hyper-echogenic” and may be represented with lighter colors on ultrasonic images. In contrast, tissues with lower echogenicity may be called “hypo-echogenic” and may be represented with darker colors. In some exemplary embodiments, echogenicity parameter may have three possible values: “Hyperechogenic”, “Isochogenic” or “Hypoechogenic”, describing the tumor within the image.

In the case of B-mode ultrasonic image of a tumor within a human breast tissue, the semantic representation parameters may consist of the BI-RADS parameter with four possible values, the homogeneity parameter with two possible values and the echogenicity parameter with three possible values, the total number of combination of values of the semantic representation parameters may be 4*2*3=24 combinations, which is below the human comprehension threshold.

In some exemplary embodiments, when the image is a mammographic image, the semantic representation parameters may consist of a BI-RADS parameter, a homogeneity parameter and a density parameter. The density parameter may refer to the density of the tumor, and may have three possible values: “radiolucent”, “radiopaque”, and “calcified”. In this case, the total number of combination of values of the semantic representation parameters of the mammographic image may be 4*2*3=24 combinations, which is below the human comprehension threshold.

In some exemplary embodiments, the image representation set that represents a specific image (e.g., valuation of all semantic representation parameters) may be used to determine a decision regarding the clinical diagnosis problem. In some exemplary embodiments, the decision may be determined by a practitioner in view of the information provided to the practitioner by a computerized device. Additionally or alternatively, the decision may be determined by an automated device, such as a Decision Support System (DSS), a Computer-Aided Diagnosis (CAD) tool, or the like.

In some exemplary embodiments, the clinical diagnosis problem may be obtained and in response to obtaining the problem, the semantic representation parameters used as the image representation set may be selected automatically. The problem may be obtained from the user or other external sources. In some exemplary embodiments, the same process may be performed with respect to various different problems, each potentially being associated with an image representation set that is comprised of a different set of semantic representation parameters.

One technical effect of utilizing the disclosed subject matter may be providing a unified language for both human practitioners and computerized devices. The unified language may be related to a specific problem that is being addressed by the human practitioner. The answer to the problem may be ascertainable from an image. The unified language may assist the human practitioners and the computerized devices to make a decision regarding the problem. In some exemplary embodiments, the human practitioner may verify that values that a computerized device determined with respect to an image is correct and thereby verify that the conclusion of the computerized device is based on correct facts. In some exemplary embodiments, in case the human practitioner identifies wrong values, the human practitioner may modify the values to correct values to provide the computerized device with the correct facts to base its conclusion on.

Another technical effect may be creating small image representation sets with a small total number of combination values of the semantic representation parameters. The small total number of combination values may be below a human comprehension threshold. A human practitioner may thus intuitively be able to identify an impact of each different valuation of the image representation set. This is as opposed to a case in which the human practitioner is faced with an image representation set that can be evaluated to thousands of different valuations and therefore it may be impossible for a human practitioner to be able to tell what the outcome of each potential valuation is. As a result, the human practitioner may determine to disregard an answer provided by the computerized device in case it is inconsistent with the human practitioners knowledge of the potential impact of the valuation. In some cases, the human practitioner may provide the impact information to a database utilized by the computerized device, and as the total number of valuations is relatively small, such a task is not especially tedious or requires automation. This may allow the computerized device to be enriched with information provided by humans and not necessarily rely on machine learning techniques which may identify features that are of importance to the answer but are not necessarily understandable by humans, By providing a small image representation sets, practical utilization of the image representation sets may be yielded.

In some exemplary embodiments, the image representation set may be used to teach or train physicians to correctly diagnose patients. The physicians may be trained by showing them different image representation sets and requiring the physicians to provide an answer to the problem based on the image representation set. In some cases, an image corresponding to the image representation set may be shown to the physicians. In some exemplary embodiments, physicians may be taught which image representation set corresponds to each problem, thereby teaching them which features to look for in an image for each problem they encounter. In some exemplary embodiments, a minimal image representation set may be used to teach physicians correct diagnosis thought processes.

Yet another technical effect may be creating an image representation set that comprise a relatively small number of semantic representation parameters, such as no more than three parameters, five parameters, seven parameters, or the like. Providing a small number of parameters may serve the purpose of having a total small number of combinations. Additionally or alternatively, a small number of parameters may be easier for a person to comprehend. For example, it may be hard for a person to remember the values of twenty different parameters describing a single image. It may also be hard for a person to identify sub-groups of parameters whose combination of values is important for a diagnosis or for providing an answer to the problem. By limiting the number of parameters, such difficulties may be overcome, while providing sufficient information by selecting specific parameters for each problem.

Yet another technical effect may be using a minimal image representation set for a problem. The minimal image representation set may consist of a minimal set of semantic representation parameters that is sufficient to distinguish between two different instances of the problem for the purpose of providing an answer to the problem. The minimal representation set may not comprise any information that is not needed for this purpose. Furthermore, the minimal set may be different for each problem (e.g., one minimal set for determining malignancy in a breast tumor that is shot using ultrasound, a different set for determining malignancy in a breast tumor that is imaged using mammography and a different set for determining malignancy in a lung tumor that is imaged using ultrasound).

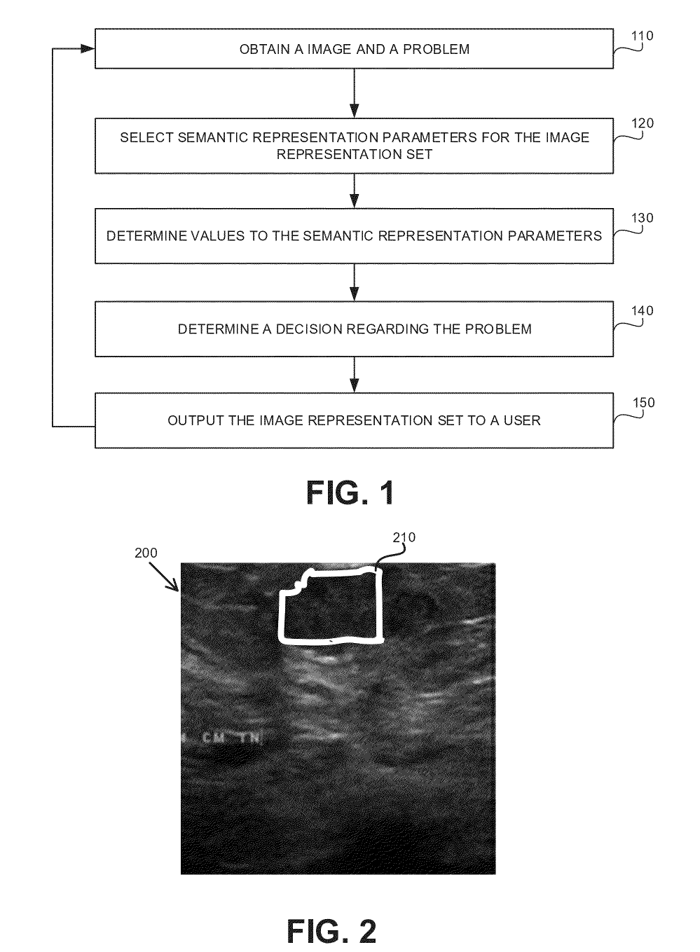

Referring now to FIG. 1 showing a flowchart diagram of a method, in accordance with some exemplary embodiments of the disclosed subject matter.

In Step 110, an image and a problem may be obtained. The image may be a medical image of a subject. The image may be produced using an imaging modality. In some exemplary embodiments, the image may be an ultrasonic B-mode image which may display a two-dimensional cross-section of an anatomical tissue being imaged. In other exemplary embodiments, the image may be a mammographic image of a human breast. The image may be obtained from a medical imaging device, may be retrieved from a repository, or the like. As an example only, the subject may be a breast tumor and the clinical diagnosis problem may be analyzing the breast tumor to be either malignant or benign.

In some exemplary embodiments, the problem may be a clinical diagnosis problem that may be ascertainable from the image.

In some exemplary embodiments, the image and the problem may be obtained from an external source. The image may be obtained from an imaging tool that produced the image. Additionally or alternatively, the image may be obtained from a repository of images. Additionally or alternatively, the image may be provided by a user. In some exemplary embodiments, the problem may be obtained from a user, may be associated with the image, such as defined by meta data of the image, inherently defined for the image (e.g., in case of mammography).

In Step 120, semantic representation parameters for an image representation set may be selected. The selection may be based on the clinical diagnosis problem. In some exemplary embodiments, the selection may be based on a type of imaging modality used to produce the image. In some exemplary embodiments, the selection may be performed automatically, such as based on rules, database, configurations, or the like. In some exemplary embodiments, a database retaining a set of parameters associated with the problem may be accessed in order to select the parameters.

In some exemplary embodiments, the semantic representation parameters may be selected from a group of potential parameters.

In some exemplary embodiments, when the image is a B-mode ultrasonic image, and the subject is a tumor within a breast tissue, the semantic representation parameters may be selected to consist of a BI-RADS parameter, a homogeneity parameter and an echogenicity parameter.

In some exemplary embodiments, when the image is a mammographic image, the semantic representation parameters may be selected to consist of a BI-RADS parameter, a homogeneity parameter and a density parameter.

In Step 130, values to the semantic representation parameters may be determined. The determination may be performed automatically by extracting a value for each parameter from the image. The value may be associated with a subject appearing in the image, such as a tumor that is seen in the image.

In Step 140, a decision regarding the problem may be determined. The decision may be determined based on the values of the image representation set of the image. In some exemplary embodiments, the decision may be determined by a decision support system, or by a similar automatic system. In some exemplary embodiments, the determination may be based on a database describing clinical data, experimental data, or the like, that can be used to ascertain an answer to the problem (e.g., providing a most likely hypothesis to an answer).

In Step 150, the image representation set may be output to a user. In some exemplary embodiments, the user may be able to verify the automatic determination of the image representation set. In some exemplary embodiments, the output may include the determined decision and the user may agree or disagree with the proposed answer.

In some exemplary embodiments, Step 110-150 may be performed multiple times with respect to different images, different problems, combination thereof, or the like. In some exemplary embodiments, an image representation set for a first problem may differ in the selected semantic representation parameters (e.g., selected in Step 120) than an image representation set for a second problem.

Referring now to FIG. 2 showing an illustration of an image, in accordance with some exemplary embodiments of the subject matter.

In some exemplary embodiments, an Image 200 may be a medical image of a Subject 210. Image 200 may be a B-mode ultrasonic image modality. Image 200 may be an image of an anatomical tissue, such as breast tissue. In some exemplary embodiments, Subject 210 may be a breast tumor.

In some exemplary embodiments, a clinical diagnosis problem about Subject 210 may be obtained. The answer to the clinical diagnosis problem may be ascertainable from Image 200. As an example, if Subject 210 is a breast tumor, the clinical diagnosis problem may be determining malignancy of the breast tumor presented as Subject 210.

In some exemplary embodiments, semantic representation parameters for an image representation set of Image 200 may be selected (e.g., such as in Step 120 of FIG. 1). The selection may be based on the clinical diagnosis problem. As an example, in case Image 200 is a B-mode ultrasonic image, and Subject 210 is a breast tumor, the semantic representation parameters may be a BI-RADS parameter, a homogeneity parameter and an echogenicity parameter. Values to the semantic representation parameters may be determined with respect to Image 200 and according to the imaging modality. The determination may be according to the clinical diagnosis problem and the imaging modality. As an example, a “4” value may be determined to the BI-RADS parameter, a “heterogeneous” value may be determined to homogeneity parameter and a “hyper-echonic” value to the echogenicity parameter.

In some exemplary embodiments, a decision regarding the clinical diagnosis problem may be determined (e.g., as described in Step 140 of FIG. 1). The decision may be determined based on the values of the image representation set of Image 200. Referring again to the same example mentioned before, the clinical diagnosis problem may be determining malignancy of the tumor, the decision according to the values of the image representation set may be that the tumor is malignant.

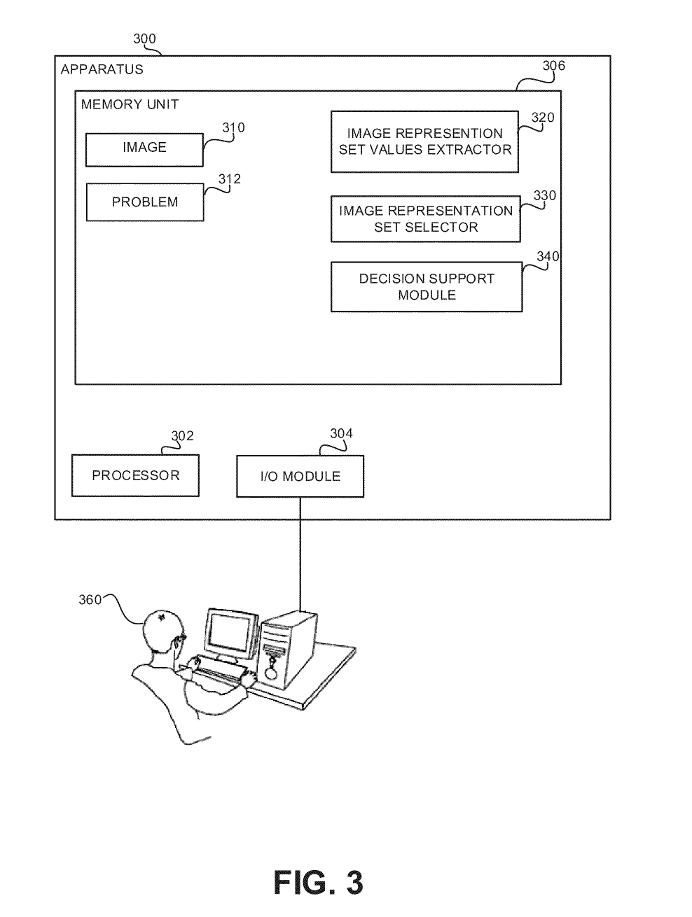

Referring now to FIG. 3 showing an apparatus, in accordance with some exemplary embodiments of the disclosed subject matter. An Apparatus 300 may be configured to determine values to an image representation set with respect to an Image 310, wherein the image representation set may consist of semantic representation parameters of Image 310 according to a Problem 312. Apparatus 300 may be configured to perform the method depicted in FIG. 1.

In some exemplary embodiments, Image 310 may be a clinical image. In some exemplary embodiments, Problem 312 may be a clinical diagnosis problem whose answer may be ascertainable from Image 310 with or without additional clinical information.

In some exemplary embodiments, Apparatus 300 may comprise a Processor 302. Processor 302 may be a Central Processing Unit (CPU), a microprocessor, an electronic circuit, an Integrated Circuit (IC) or the like. Processor 302 may be utilized to perform computations required by Apparatus 300 or any of it subcomponents.

In some exemplary embodiments of the disclosed subject matter, Apparatus 300 may comprise an Input/Output (I/O) Module 304. I/O Module 304 may be utilized to provide an output to and receive input from a User 360. It will be noted that User 360 may or may not be an expert in the field of analyzing of Image 310, such as a radiologist, a physician, or the like. In some exemplary embodiments, Apparatus 300 may operate without having a user. I/O Module 304 may be used to obtain Image 310 and Problem 312. I/O Module 304 may be used to output a decision regarding Problem 312, to output the valuation of the image representation set, or the like.

In some exemplary embodiments, Apparatus 300 may comprise a Memory Unit 306. Memory Unit 306 may be a hard disk drive, a Flash disk, a Random Access Memory (RAM), a memory chip, or the like. In some exemplary embodiments, Memory Unit 306 may retain program code operative to cause Processor 302 to perform acts associated with any of the subcomponents of Apparatus 300.

In some exemplary embodiments, Image 310 may be a clinical image of an anatomical tissue. Image 310 may be produced using an imaging modality. As an example, Image 310 may be an ultrasonic image. Additionally or alternatively, Image 310 may be a B-mode ultrasonic image, such as 200 of FIG. 2, produced by a brightness-mode (B-mode) ultrasound imaging modality. Additionally or alternatively, Image 310 may be a mammographic image.

In some exemplary embodiments of the disclosed subject matter, Apparatus 300 may comprise an Image Representation Set Values Extractor 320. Image Representation Set Values Extractor 320 may be configured to determine values to an image representation set with respect to Image 310. The image representation set may consist of semantic representation parameters of Image 310 according to the imaging modality of Image 310 and according to Problem 312.

In some exemplary embodiments, an Image Representation Set Selector 330 may be configured to select one or more semantic representation parameters to be used as an image representation set. In some exemplary embodiments, the selection may be performed automatically, such as based on Problem 312. In some exemplary embodiments, the automatic selection may be different for images of different modalities, for different problems, or the like. Additionally or alternatively, the selection may be predetermined, based on user input, or the like.

In some exemplary embodiments of the disclosed subject matter, Apparatus 300 may comprise a Decision Support Module 340. Decision Support Module 340 may determine the decision regarding Problem 312 based on the values of the image representation set of Image 310. In some exemplary embodiments, the decision may be determined using an expert database, such as inputted by experts based on experimental information, historical clinical data, studies, or the like.

In some exemplary embodiments, Decision Support Module 340 may determine the decision using automatic training, such as performed as part of machine learning techniques. A training database may be provided and Decision Support Module 340 may be configured to learn to predict a solution for a problem (e.g., Problem 312) based on the available features (e.g., valuation of the image representation set).

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

What is claimed is:

1. A computer-implemented method comprising:
obtaining an image of a subject, wherein the image is produced using an imaging modality;
automatically determining, by a processor, values to an image representation set with respect to the image, wherein the image representation set consists of semantic representation parameters of the image according to the imaging modality and according to a clinical diagnosis problem that is ascertainable from the image, wherein a total number of combinations of values of the semantic representation parameters is below a human comprehension threshold; and
determining a decision regarding the clinical diagnosis problem based on the values of the image representation set of the image.

2. The computer-implemented method of claim 1 further comprising:
obtaining the clinical diagnosis problem;
automatically selecting the semantic representation parameters for the image representation set based on the clinical diagnosis problem.

3. The computer-implemented method of claim 2 further comprising:
obtaining a second image of a second subject;
obtaining a second clinical diagnosis problem with respect to the second subject, wherein the second clinical diagnosis problem is ascertainable from the second image;
automatically selecting semantic representation parameters for a second image representation set based on the second clinical diagnosis problem, wherein the semantic representation parameters for the second image representation set are different, at least in part, from the semantic representation parameters for the image representation set; and
automatically determining values for the second image representation set with respect to the second image.

4. The computer-implemented method of claim 1 further comprising outputting to a user the image representation set, whereby the user is enabled to verify the automatic determination of the image representation set.

5. The computer-implemented method of claim 1, wherein the subject is a tumor within an anatomical tissue, wherein the clinical diagnosis problem is determining malignancy of the tumor.

6. The computer-implemented method of claim 5, wherein the image is an ultrasonic image, wherein the imaging modality is a brightness-mode ultrasound imaging modality, wherein the subject is a tumor within a breast tissue, wherein the semantic representation parameters consist of a Breast Imaging-Reporting and Data System (BI-RADS) parameter, a homogeneity parameter and an echogenicity parameter.

7. The computer-implemented method of claim 5, wherein the image is a mammographic image, wherein the semantic representation parameters consist of a Breast Imaging-Reporting and Data System (BI-RADS) parameter, a homogeneity parameter and a density parameter.

8. The computer-implemented method of claim 1, wherein said determining comprises determining the decision by a decision support system.

9. The computer-implemented method of claim 1, wherein the total number of combinations of values of the semantic representation parameters is greater than ten and below a hundred.

10. The computer-implemented method of claim 9, wherein the total number of combinations of values of the semantic representation parameters is greater than a dozen and below forty.

11. A computerized apparatus having a processor, the processor being adapted to perform the steps of:
obtaining an image of a subject, wherein the image is produced using an imaging modality;
determining values to an image representation set with respect to the image, wherein the image representation set consists of semantic representation parameters of the image according to the imaging modality and according to a clinical diagnosis problem that is ascertainable from the image, wherein a total number of combinations of values of the semantic representation parameters is below a human comprehension threshold; and
determining a decision regarding the clinical diagnosis problem based on the values of the image representation set of the image.

12. The computerized apparatus of claim 11, wherein the processor is further adapted to perform the steps of:
obtaining the clinical diagnosis problem;
selecting the semantic representation parameters for the image representation set based on the clinical diagnosis problem.

13. The computerized apparatus of claim 12, wherein the processor is further adapted to perform the steps of:
obtaining a second image of a second subject;
obtaining a second clinical diagnosis problem with respect to the second subject, wherein the second clinical diagnosis problem is ascertainable from the second image;
selecting semantic representation parameters for a second image representation set based on the second clinical diagnosis problem, wherein the semantic representation parameters for the second image representation set are different, at least in part, from the semantic representation parameters for the image representation set; and
determining values for the second image representation set with respect to the second image.

14. The computerized apparatus of claim 11, wherein the processor is further adapted to output to a user the image representation set, whereby the user is enabled to verify the automatic determination of the image representation set.

15. The computerized apparatus of claim 11, wherein the subject is a tumor within an anatomical tissue, wherein the clinical diagnosis problem is determining malignancy of the tumor.

16. The computerized apparatus of claim 15, wherein the image is an ultrasonic image, wherein the imaging modality is a brightness-mode ultrasound imaging modality, wherein the subject is a tumor within a breast tissue, wherein the semantic representation parameters consist of a Breast Imaging-Reporting and Data System (BI-RADS) parameter, a homogeneity parameter and an echogenicity parameter.

17. The computerized apparatus of claim 15, wherein the image is a mammographic image, wherein the semantic representation parameters consist of a Breast Imaging-Reporting and Data System (BI-RADS) parameter, a homogeneity parameter and a density parameter.

18. The computerized apparatus of claim 11, wherein the total number of combinations of values of the semantic representation parameters is greater than ten and below a hundred.

19. The computerized apparatus of claim 18, wherein the total number of combinations of values of the semantic representation parameters is greater than a dozen and below forty.

20. A computer program product comprising a computer readable storage medium retaining program instructions, which program instructions when read by a processor, cause the processor to perform a method comprising:
obtaining an image of a subject, wherein the image is produced using an imaging modality;
determining values to an image representation set with respect to the image, wherein the image representation set consists of semantic representation parameters of the image according to the imaging modality and according to a clinical diagnosis problem that is ascertainable from the image, wherein a total number of combinations of values of the semantic representation parameters is below a human comprehension threshold; and
determining a decision regarding the clinical diagnosis problem based on the values of the image representation set of the image.
